(19) United States
(12) Patent Application Publication
(10) Pub. No.: US 2024/0238542 A1
(43) Pub. Date: Jul. 18, 2024

(54) MULTI-DOSE MEDICAMENT DELIVERY DEVICE

(71) Applicant: Therkind Limited, London (GB)

(72) Inventors: Philip Wilson Braithwaite, Cheltenham (GB); David James Purdie, Cambridge (GB); Andrew John Ede, Cambridge (GB); Nicholas Andrew Earl, Cambridge (GB); Elliot Goodman, London (GB); Louise Rawcliffe, London (GB)

(73) Assignee: Therkind Limited, London (GB)

(21) Appl. No.: 18/621,614

(22) Filed: Mar. 29, 2024

Related U.S. Application Data
(63) Continuation-in-part of application No. 16/638,534, filed on Feb. 12, 2020, now Pat. No. 11,969,547.

(30) Foreign Application Priority Data
Aug. 30, 2017 (GB) 1713899.1

(51) Int. Cl. A61M 15/08; A61M 15/00

(52) U.S. Cl. CPC A61M 15/08; A61M 15/0028; A61M 2202/064; A61M 2205/073

(57) ABSTRACT

There is described a multi-unit dose dry powder medicament delivery device (1) comprising: a first element (2) comprising a single use nozzle (3) located in a body (4) which is at least partially lined with an inner member, an inner sleeve (6) comprising an airway (5) and a cartridge seat (11); and a second element (20) adapted to be releasably attached to the first element (2), said second element (20) comprising an actuator provided with an air source (23) and a valve (22); wherein the medicament cartridge comprises a replaceable cartridge, e.g. a replaceable pre-dosed cartridge.

### CROSS REFERENCE TO RELATED APPLICATIONS

This application is a Continuation-in-Part of U.S. patent application Ser. No. 16/638,534 filed Feb. 12, 2020 and the benefit from International Patent Application No. PCT/GB2018/052453 filed Aug. 30, 2018 which claims priority to United Kingdom Patent Application No. 1713899.1 filed Aug. 30, 2017, the entirety of which applications are incorporated by reference herein.

### FIELD OF THE INVENTION

The present invention relates to novel multi-dose dry powder medicament delivery device, uses thereof and methods of manufacture.

More particularly, the invention relates to novel multi-dose/multi-use dry powder medicament delivery device which is suitable for use as, for example, a nasal medicament delivery device for the delivery of medicaments, e.g. for the treatment of a respiratory disorder and especially for the delivering of a vaccine or a hormone, such as, glucagon, in dry powder form.

The medicament delivery device is also suitable for use for the delivery of a medicament in, e.g. in powder form, into an infusion bag, the medicament then being delivered to the patient as a fluid infusion, for example, as an ‘IV drip’.

### BACKGROUND TO THE INVENTION

In recent years drug formulations have been developed in dry powder form, e.g. for delivery by inhalation, or by admixing in a solution for delivery by intravenous infusion. Such dry powder formulations include existing compounds reformulated into dry powder form and newly developed compounds, used in the treatment of many conditions and diseases.

Drug formulations in the form of inhaled dry powders offer advantages over other forms such as liquids and tablets, particularly when considering storage and stability.

Oral or nasal delivery of a medicament using a dry powder medicament delivery device is a particularly attractive method of drug administration as such devices can be relatively easy for a patient to use. As well as delivering medicament to treat local diseases of the airway and other respiratory disorders, dry powder medicament delivery devices have more recently also be used to deliver drugs to the bloodstream via the lungs or nasal passages, thereby avoiding, for example, the need for injections.

One advantage of delivering a drug in a dry powder form is that very low dosages of the medicament may be used. However, in many situations the disadvantage of using dry powder formulations lies in the complication of actual delivery to the patient's area of treatment and/or the metering of very low dosages of medicament. The principle problem lies in the accurate metering and containment of a unit of dose and subsequent appropriately controlled release or dispensing of the unit dose. This is a significant impediment for the pharmaceutical industry in developing the potential of dry powder formulations to end products.

Dry powder delivery devices are most commonly known in the form of dry powder inhalers, these range from metered dose devices such as Clickhaler® where the dry powder medicament is stored in a reservoir and metered by operation of the device; to unit dose devices where the medicament is stored in individual unit doses in capsules (e.g. Spinhaler®) or foil blisters (e.g. Diskhaler®). These devices are generally cumbersome and complex in construction and, although suitable for their intended use for delivering medicament to the lung, their adaption for delivery to other areas of treatment, such as the nasal passage and/or nasal cavity, is generally unsatisfactory.

The present invention seeks to provide a dry powder medicament delivery device that overcomes or substantially alleviates the problems with conventional inhalation devices and/or infusion devices. In particular, the invention seeks to provide a device having a significantly simpler construction than known devices. The dry powder medicament delivery device of the present invention is also easier to manufacture, assemble and operate, as well as being cheaper to manufacture. The present invention also seeks to provide a device that has the ability to re-use the air source and valve, and accommodate a single use nozzle and drug cartridge, thereby reducing cost and eliminating waste.

International Patent application No. WO 2013/088112 describes a single dose disposable dry powder medicament delivery device comprising a medicament container containing a unit dose of dry powder medicament, a medicament dispensing assembly and an air source. However, in the development of the aforementioned single dose disposable dry powder medicament delivery device the requirement for further improvements emerged.

Furthermore, recently dry powder intranasal vaccines have been developed. There are a number of advantages to intranasal delivery of drugs, in particular in dry powder form. Intranasal drug delivery offers rapid uptake into the blood stream by absorption through the nasal mucosa, the potential to reduce or eliminate cold chain management of vaccines during storage and transportation, and the elimination of needles and the potential for needle stick injuries.

Intranasal vaccination represents an attractive non-invasive alternative to needle-based injection and provides superior protection at mucosal surfaces. However, new formulations and delivery devices are needed to improve efficacy and reduce the refrigerated storage and distribution requirements associated with standard liquid vaccines.

Vaccines formulated as liquids can be subject to chemical degradation, e.g., aggregation, denaturation, hydrolysis, and oxidation that can result in their inactivation. Liquid vaccine formulations can also be sensitive to temperature: high temperatures can increase inactivation, and freezing temperatures can result in ice that can damage antigen in the vaccine. Thus, to prevent inactivation, liquid vaccines often must be stored at a temperature range of from 2-8° C.

The mode of administration of a vaccine can play a role in its efficacy. One mode of administration, non-parental administration (e.g., nasal), can induce and promote mucosal and systemic immune responses. In addition, nasal mucosa can help bind a virus or other pathogen at the mucosal surface, preventing access of the pathogen to deeper tissues and/or decreasing the likelihood of full-blown infection.

### SUMMARY OF THE INVENTION

As described herein, the principal improvement over the device described in International Patent application No. WO 2013/088112 was to develop a device that offered the ability to reuse the air source and the delivery device; and accommodate a single use drug cartridge with a reusable nozzle, thereby reducing cost and eliminating waste.

Therefore, according to a first aspect of the invention there is provided a multi-unit dose dry powder medicament delivery device comprising:
a first element comprising a single use nozzle located in a body which is at least partially lined with an inner sleeve, said inner sleeve comprising an airway and a cartridge seat; and
a second element adapted to be releasably attached to the first element, said second element comprising an actuator provided with an air source and a valve;
wherein the medicament cartridge comprises a replaceable cartridge, e.g. a replaceable pre-dosed cartridge.

The multi-unit dose dry powder medicament delivery device of the invention will generally be used in conjunction with a cartridge comprising a dry powder medicament.

The medicament cartridge may comprise a unit dose of drug or medicament. The medicament cartridge comprises an elongate member provided with an inset cavity which acts as a medicament reservoir. The medicament cartridge will desirably be provided with a closure sleeve, said closure sleeve being slidably mounted around the inset cavity.

In a particular aspect of the invention the medicament cartridge comprises a replaceable cartridge, e.g. a replaceable pre-dosed cartridge. The use of a replacement cartridge is advantageous in that, inter alia, differently dosed cartridges to enable different dose quantities from the same device.

Each cartridge is made up of three components; a medicament container (or carrier), a sleeve and a foil cover. In this aspect of the invention the carrier will be a medicament carrier which will generally comprises a container carrying a medicament dose. The sleeve will substantially surround or house the medicament carrier. The foil cover will generally be positioned over one or both ends of the sleeve. Prior to use the foil cover will be fully intact—indicating that the cartridge is useable. In use, the foil cover is ruptured or perforated, which also provides an indication to the user that the cartridge has been used and should not be used again.

When present, the foil cover is primarily intended to ensure that, when the delivery device is in use, the pressure built up inside the air chamber does not dislodge the carrier before it is physically moved by a spigot at the point where there is a minimum level of pressure built to ensure pickup. The foil is not intended to add any additional protection for the powder in terms of stability. However, the foil cover may act as a valve.

An important aspect of the airway of the inner sleeve is that it comprises at least one baffle element such that, in use, the medicament powder is caused to flow in a non-linear pathway before being expelled from the nozzle of the delivery device. Indeed, the airway of this aspect of the invention is such that the air is caused to substantially flow via at least two angular turns, e.g. right angled turns, i.e. a first right angle turn followed by a second right angle turn as it is expelled from the delivery device. Preferably, two angular turns are present. This provides a significant advantage in efficiently deagglomerating the powder whilst not impeding the efficient clearing of the entire dose from the drug cavity of the cartridge with sufficient velocity for the powder to reach its intended target, for example, the nasal cavity.

The elongate member of the medicament cartridge and slidable outer sleeve together provide a simple secure storage compartment for the powdered medicament which, when activated, also provide an effective means of powder deagglomeration as herein described.

However, although a cartridge comprising an elongate member and slidable outer sleeve is to be preferred, it will be understood by the person skilled in the art that a variety of known unit dose medicament containers may suitably be used in the dry powder medicament delivery device. Thus, for example, the unit dose dry powder formulation may comprise a pre-packaged capsule or blister, which each contains an individual dose, usually in the form of dose of the powder, which has been accurately and consistently measured. The use of a pre-packaged capsule or blister may be suitable if combined with a shuttle valve as herein described.

As herein described a medicament dispensing assembly generally comprises a body and a nozzle. More particularly, the medicament dispensing assembly comprises a body, a nozzle and an airway, the airway being provided with an inlet and an outlet, e.g. an air inlet and an air/medicament outlet. For an intranasal delivery device, the nozzle is desirably sized and shaped to be suitable for placing inside a patient's nostril. Although it will be understood by the person skilled in the art that the medicament dispensing assembly may also be designed so as to be suitable for oral delivery, such as an inhaler for delivery to the lungs (respiratory tract).

The releasable attachment of the first element to the second element may comprise a snap-fit, screw fit, bayonet fit, a hinged clamp and the like. In a preferred aspect of the invention the releasable attachment comprises a screw fit.

In a further aspect of the invention the inner sleeve may be provided with a shuttle valve. A shuttle valve will be dimensioned such that in the closed position it blocks the airway only until the device is primed by a user. The shuttle valve may be provided with a pair of orifices which, when the delivery device is primed, align with the airway of the device. The pair of orifices will generally comprise an entrance orifice, enabling air to be drawn into the medicament carrier; and an exit orifice, allowing air and the entrained medicament, to be drawn out of the device. The use of a shuttle valve is advantageous in that it can reduce the potential for medicament powder spillage if the device is inverted or shaken.

The use of a shuttle valve acts to block the airways in step (i); in step (ii) the shuttle valve is released from its detent, but the airways remain closed by the valve; when the device is actuated in step (iii) the shuttle valve moves to an “open” position and the airways are unblocked.

The actuator in the second element may comprise a spigot element which, when inserted into the first element, pushes the medicament container into position for delivery of the medicament. Preferably the spigot element is provided with at least one aperture to enable air to pass from the air source and valve through the spigot.

The actuator may comprise a plunger, such that, in use, a user presses or depresses the plunger in order to actuate the device and urge the spigot toward the assembly.

In addition, the delivery device may be provided with a removable protective cap or cover adapted to be positioned over the plunger. When the protective cap is positioned over the plunger it prevents accidental actuation of the device, for example, when loading the cartridge and/or closing the lid. In addition, the protective cap or cover provides a further benefit of keeping dirt and dust out of the air chamber.

In one aspect of the invention the protective cap may be held in the closed (protective) position by the use of biasing means. In one aspect the biasing means comprises a torsion spring, such that a user must actively hold the protective cap out of the way in order to access the plunger. In another aspect of the invention the protective cap may be provided with an integral moulded hinge which acts to urge the protective cap to the closed (protective) position. The protective plunger cap also provides physical protection for the device if it is dropped, but does not affect the operation of the device if the plunger cap is damaged.

In another aspect of the invention the actuator or plunger may be provided with a latch mechanism, such that the latch retains the plunger in place after the device has been actuated until the device is reset by, for example, by opening up the plunger cover in order to remove the used medicament cartridge.

Thus, the plunger latch may comprise a resilient protrusion adapted to releasably engage with a portion of the first element of the delivery device. In particular, the first element of the device may be provided with one or more grooves adapted to releasably engage with the plunger latch.

In use, once the protective plunger cover is opened or removed, the plunger latch clears the retaining feature and is driven back to the neutral position by the spring.

The use of a plunger latch may be advantageous because it provides a clear indication to the user that the device has been actuated correctly.

In another aspect of the invention the delivery device comprises a dynamic relationship between the spigot, the entrance orifice and the medicament carrier. For example, in use, as the spigot progressively moves the carrier forward within the sleeve, the entrance orifice is progressively revealed. The dynamic nature of the progressive revealing or opening of the entrance orifice improves air flow through the carrier resulting in improved pick-up of the medicament from the medicament container and avoids dead spots (where medicament powder can remain without being dispensed) by generating a small amount of turbulence within the container and provides dynamic air flow.

The air source may comprise a delivery pump e.g. it may comprise a syringe. Such a syringe may, for example, comprise a conventionally known propriety syringe, e.g. a disposable syringe, or may comprise a conventionally known means for ejecting an air stream, for example, an insufflator or other such apparatus suitable for blowing air or gas upon activation, such as a cylinder and piston, bellows, a squeezable bulb or a compressed air or gas source such as a compressed air canister or from a compressed air system fitted with suitable volume and pressure control apparatus. In a preferred aspect of the invention the air source comprises bellows.

The use of a valve in the second element of the delivery device of the invention is advantageous in that it allows the volume, pressure and velocity of air that passes through the device to be controlled. The valve used in the delivery device of the present invention is a burst valve. In a preferred aspect of the invention a particular burst valve is used which is novel per se.

Thus, according a further aspect of the invention there is also provided a novel burst valve, suitable for use with a dry powder delivery device, said burst valve comprising a moulded flexible polymer disc provided with a circumferential flange to provide a sealing ring. Substantially in the centre of the disc there is provided a pyramid structure which forms a closure. The pyramid structure may comprise any number of sides. Preferably, the pyramid structure may comprise 3 to 8 sides, preferably 3 to 5 sides and especially 3 or 4 sides. A sided pyramid is most preferred. In a particular aspect of the invention the centre of disc is provided with a frustum of a pyramid as herein described, e.g. a frustum of a pyramid.

The pyramid structure is provided with at least one slit cut from the pyramid. Preferably, the at least one slit is cut along a diagonal of the pyramid. For the avoidance of doubt, the slit is cut as opposed to formed, because it should be understood that the slit remains closed in its dormant state. The pyramid structure may be provided with more than on slit, e.g. two slits, i.e. a slit along each of two diagonals, but a single slit is preferred.

The moulded flexible polymer may comprise a silicon polymer, but a preferred polymer is a Thermoplastic Elastomer (TPE) which is flexible, resilient non-porous and elastomeric.

The use of a thermoplastic elastomer is advantageous due to the ability of a thermoplastic elastomer to stretch to moderate elongations and return to its near original shape creating a longer life and better physical range than other materials. Thermoplastic elastomers generally have excellent flexural fatigue resistance, good tear & abrasion resistance, high impact strength, resistance to chemicals and are recyclable.

Exemplary commercially available TPEs include, but shall not be limited to, Pebax®, Arnitel®, Riteflex®, Enflex®, Ensoft®, Sconablend® and Ravathane®.

The use of a burst valve may be advantageous in that by positioning a dispersible dry powder material, e.g. a medicament material, downstream of a burst valve; causing the valve to open will produce a rapid depletion of the air pressure up-stream of the valve through the air-path in the device that disperses and/or deagglomerates the material. A burst valve may be fitted between the air source and the (intranasal) delivery device so that when the valve opens at a certain predetermined air pressure the velocity of the air rushing through the device will clear it very effectively.

According to the invention there is provided a method of producing an aerosol of a dry powder which comprises positioning a dispersible dry powder material, e.g. a medicament material, downstream of a burst valve member; wherein causing the valve member to flex and open will produce a rapid depletion of the air pressure up-stream of the valve or diaphragm through the air-path in the device that disperses and/or deagglomerates the material.

In one embodiment, the dry powder medicament delivery device comprises a medicament delivery device, e.g. a nasal inhaler. However, in a further embodiment drug carrier is utilised in a device to facilitate application of the drug in dry powder form via an air flow to the vagina or rectum, such a device may be fitted with a means to dilate the vagina or rectum.

The dry powder medicament delivery device according to this aspect of the invention may be suitable for delivery of a variety of medicaments and may be suitable for use in the treatment of a variety of disorders.

Thus, for example, for use as an inhaler, e.g. an inhaler for oral drug delivery, or a nasal dry powder medicament delivery device, e.g. a nasal inhaler, a variety of medicaments may be administered. Such medicaments are generally suitable for the treatment of asthma, COPD and respiratory infections. Such medicaments include, but are not limited to β2-agonists, e.g. fenoterol, formoterol, pirbuterol, reproterol, rimiterol, salbutamol, salmeterol and terbutaline; non-selective beta-stimulants such as isoprenaline; xanthine bronchodilators, e.g. theophylline, aminophylline and choline theophyllinate; anticholinergics, e.g. ipratropium bromide; mast cell stabilisers, e.g. sodium cromoglycate and ketotifen; bronchial anti-inflammatory agents, e.g. nedocromil sodium; and steroids, e.g. beclomethasone dipropionate, fluticasone, budesonide, flunisolide and ciclesonide, and isomers and/or salts or derivatives thereof.

Specific combinations of medicaments which may be mentioned include combinations of steroids, such as, beclomethasone dipropionate and formoterol; beclomethasone dipropionate and salmeterol; fluticasone and formoterol; fluticasone and salmeterol; budesonide and formoterol; budesonide and salmeterol; flunisolide and formoterol; and flunisolide and salmeterol. It is also within the scope of this invention to include combinations of one or more of the aforementioned steroids with one or more of the aforementioned β2-agonists.

However, there is increasing interest in the pulmonary delivery or intravenous delivery of medicaments due, inter alia, to the rapid onset of their efficacious effect. Thus, further medicaments which may be mentioned include systemically active materials, such as, proteinaceous compounds and/or macromolecules, for example, hormones and mediators, such as insulin, glucagon, human growth hormone, leuprolide and alpha interferon, growth factors, anticoagulants, immunomodulators, cytokines and nucleic acids. Other medicaments which may be mentioned are those for the treatment of neurological disorders, such as Parkinsonism, such as, levodopa, carbidopa, benserazide, selegiline, tolcapone, entacapone, bromocriptine, lysuride, pergolide, ropinirole and cabergoline; or migraine, such as divalroex sodium, ergotamine, methysergide, metoprolol, propranolol, zolmitriptan, vigabatrine, clonidine, ganaxolone, lysine acetylsalicylate, sumatriptan, naratriptan, timolol, almotriptan, cyproheptadine, rizatriptan, timotol, dotarizine, dihydroergotamine, metysergide, pizotifen, eletriptan, prochlorperazine, nadolol and frovatriptan. In addition, medicaments for the treatment of sexual dysfunction may be mentioned. Such disorders include erectile dysfunction where treatments include administration of phosphodiesterase type-5 (PDTE5) inhibitors, such as tadalafil, vardenafil and sildenafil; and premature ejaculation, where treatments include administration of selective serotonin reuptake inhibitors, such as dapoxetine.

However, one particular aspect of the present invention provides the dry powder medicament delivery device as herein described as a nasal dry powder medicament delivery device. A nasal dry powder medicament delivery device according to this aspect of the invention may be suitable for the delivery of any of the medicaments herein described.

An important use of the delivery device of the present invention is for the delivery of an emergency therapy. The intranasal or intravenous route of drug delivery can afford rapid absorption of drugs into the blood circulation. For example, the intranasal route can also offer a less invasive route of drug administration compared with some other routes such intravenous or intramuscular injection. Such rapid and effective drug delivery can be useful in the treatment of crisis situations such as pain, convulsions, serious hypoglycaemic reaction, etc.

The delivery device of the present invention is found to be especially advantageous in the delivery of a medicament in an emergency situation, for example, where the patient is unconscious. The nasal delivery device of the invention is especially useful in a situation where medical staff are not available. One specific such therapy is the intranasal administration of a therapeutically effective amount of glucagon to a diabetic who is experiencing a serious hypoglycaemic reaction. Glucagon is a hormone that causes the liver to release glucose into the blood and is used to quickly increase blood sugar levels in diabetics with hypoglycaemia (low blood sugar). Glucagon is usually provided in powder form and given as an injection either into a vein, an arm or leg muscle or under the skin as directed, usually to an unconscious patient. The glucagon powder must first be dissolved in a diluting fluid and must be used immediately after it has been mixed.

Therefore, according to a particular aspect of the present invention there is provided a method of delivering glucagon to a patient which comprises the use of a medicament delivery device as herein described, especially the use of a dry powder nasal dry powder medicament delivery device.

A further category of patients for whom intravenous or intramuscular injection may be problematical is infants and young children, therefore the use of the delivery device of the present invention as an intranasal, oral or rectal drug delivery device may be beneficial.

In addition, when used as an inhaler, e.g. an oral inhaler and especially a nasal dry powder medicament delivery device, the medicament delivery device as herein described may suitably be used for the delivery of one or more dry powder vaccines.

Dry powder vaccine compositions for intranasal delivery are described in International Patent application No. WO 2011/129120. Therefore, a dry powder vaccine for use in association with a medicament delivery device, such as a nasal dry powder medicament delivery device, of the present invention, can be useful for the prevention and/or treatment of infection by any virus.

However, it will be understood by the person skilled in the art that the dry powder medicaments mentioned herein can be delivered using the delivery device of the present invention, to deliver a dry powdered medicament to, for example, an intra-venous infusion bag.

In a further embodiment a medicament carrier may be utilised in a device to facilitate application of the medicament in dry powder form, by entraining the powder in a gel for administration to a body orifice.

In a yet further embodiment a medicament carrier may be utilised in a device to facilitate dermal or transdermal application of the drug in dry powder form, via a gel applicator.

It will be appreciated that the above descriptions can apply to the treatment of animals as well as humans.

The invention further provides a method of delivering a medicament, e.g. a dry powder medicament, to a patient which comprises the use of a dry powder medicament delivery device as herein described.

Fluid may be prevented from leaking from the device by a seal which has a circumferential seal feature to seal against a seal housing within the main body and a face seal feature to seal against the face of the medicament carrier.

The invention further provides a method of treatment of a patient with a disorder which comprises the administration of a medicament using a medicament delivery device as herein described.

The method of treatment according to this aspect of the invention may comprise the administration of any one or more of the therapeutically active agents described herein. However, there is especially provided a method of delivering a vaccine, e.g. a dry powder vaccine to a patient.

More especially, the invention provides a method of treating a patient which comprises the administration of a therapeutically effective amount of glucagon to a diabetic experiencing a hypoglycaemic reaction.

Desirably the inner sleeve comprises a suitable first plastics material and the medicament cartridge, comprising an elongate member provided with an inset cavity, comprises an alternative second plastics material. The preferred device, i.e. comprising first and second plastics materials may be manufactured by a variety of methods, including a method known as two shot moulding or injection moulding.

The term ‘suitable’ plastics material as herein described is intended to mean, for example, first and second plastics material that do not to bond to each other. The lack of bonding between the first and second plastics material allows the sleeve to slide over the elongate member.

Thus, according to a further aspect of the invention there is provided a method of two shot moulding a medicament container comprising an elongate member comprising a suitable first plastics material and the slidable sleeve comprises an alternative second plastics material.

The unit dose medicament container as herein described is advantageous in that, inter alia, it is easy and economic to manufacture and can be easily filled either on an individual basis or in a fast moving production line. One method of filling is described in the specific embodiments herein.

According to a further aspect of the invention there is provided a dry powder medicament delivery device kit comprising:
a first element comprising a single use nozzle, said nozzle being located on a body which is at least partially lined with an inner sleeve, said inner sleeve comprising an airway and a cartridge seat;
a second element adapted to be releasably attached to the first element, said second element comprising an actuator provided with an air source and a valve;
wherein the medicament cartridge comprises a replaceable cartridge; and at least one cartridge comprising a dry powder medicament.

The kit according to this aspect of the invention which includes a plurality of medicament cartridges.

### BRIEF DESCRIPTION OF THE DRAWINGS

The invention will now be described by way of example only and with reference to the accompanying drawings in which:

FIG. 1 is a cross-sectional view of a nozzle with an inner sleeve and cartridge assembly;

FIG. 2 is a cross-sectional view of a nozzle with an inner sleeve and a cartridge assembly inserted into the nozzle;

FIG. 3 is a cross-sectional view of a first element nozzle assembly and a second element comprising an actuator provided with an air source and a valve;

FIGS. 4a, 4b and 4c are cross-sectional views of a first element nozzle assembly attached to a second element and engaging with the cartridge assembly;

FIG. 5 is a cross-sectional view of a first element nozzle assembly including a shuttle valve and a cartridge assembly with a slidable outer sleeve;

FIG. 6 is a cross-sectional view of a first element nozzle assembly including a shuttle valve and a cartridge assembly with a slidable outer sleeve inserted into the nozzle;

FIG. 7 is a cross-sectional view of a first element nozzle assembly including a shuttle valve and a second element comprising an actuator provided with an air source and a valve;

FIGS. 8a, 8b and 8c are cross-sectional views of a first element nozzle assembly including a shuttle valve attached to a second element and engaging with the cartridge assembly;

FIG. 9 is a cross-sectional view of the complete assembly after activation;

FIG. 10 is a perspective view of a pyramid valve in the closed position;

FIG. 11 is a cross-sectional view of a pyramid valve in the closed position;

FIG. 12 is a perspective view of a pyramid valve in the open position;

FIG. 13 is a cross-sectional view of a pyramid valve in the open position;

FIG. 14 is a perspective view of a complete assembly provided with a plunger cap;

FIG. 15 is a cross-sectional view of a complete assembly provided with a plunger cap;

FIGS. 16 (a) and (b) are perspective views of a device provided with a replaceable cartridge (a) before use and (b) after use;

FIGS. 17 (a) and (b) are perspective views of an assembly provided with a replaceable cartridge;

FIG. 18 is a cross-sectional view of a replaceable cartridge;

FIGS. 19 (a), (b) and (c) are cross-sectional views of a device provided with a latch;

FIGS. 20 (a) and (b) are perspective views ((a) front and (b) side) of a device provided with a plunger cap in the closed position;

FIG. 20 (c) is a rear view of a device with a plunger cap in the closed position;

FIG. 20 (d) is a perspective view of a device with a plunger cap in the open position;

FIG. 21 is a perspective view of a plunger cap with a moulded hinge; and

FIGS. 22 (a) and (b) are cross-sectional views of a device provided with a plunger latch.

### DETAILED DESCRIPTION OF THE DRAWINGS

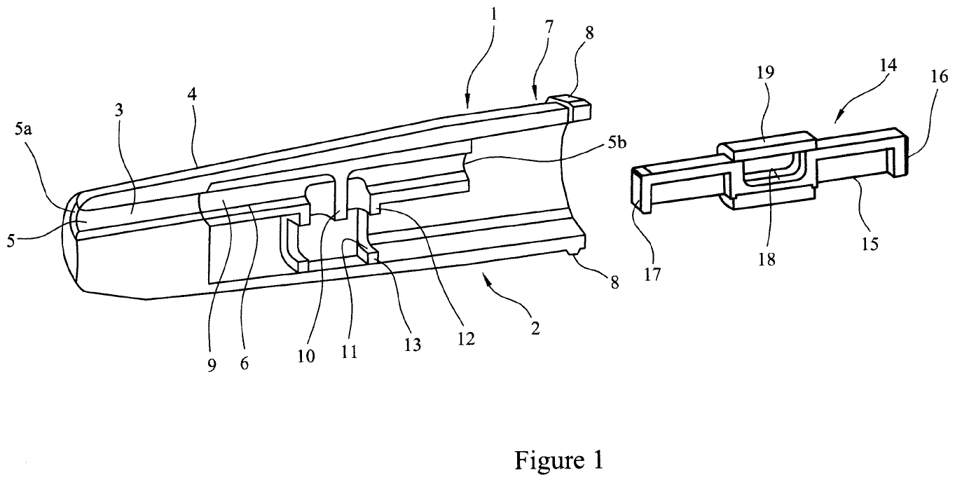

Referring to FIG. 1 a multi-unit dose dry powder medicament delivery device 1 comprises a first element 2. The first element 2 comprises a nozzle 3 located in a body 4, the nozzle 3 being provided with an airway 5 and an inner sleeve 6. At one end 7 the body 4 is provided with a circumferential rim 8. The airway 5 has an outlet 5a and an inlet 5b. The inner sleeve 6 is provided with an airway 9, which corresponds with the airway 5 of the nozzle. The airway 9 of the inner sleeve 6 is provided with a baffle 10 and a cartridge assembly seat 11 and cartridge facing lips 12 and 13.

A cartridge assembly 14 is also shown, said cartridge assembly comprising an elongate member 15 with a first end wall 16 and a second end wall 17. The elongate member 15 is provided with a cavity 18 for housing a medicament (not shown) and a closure sleeve 19.

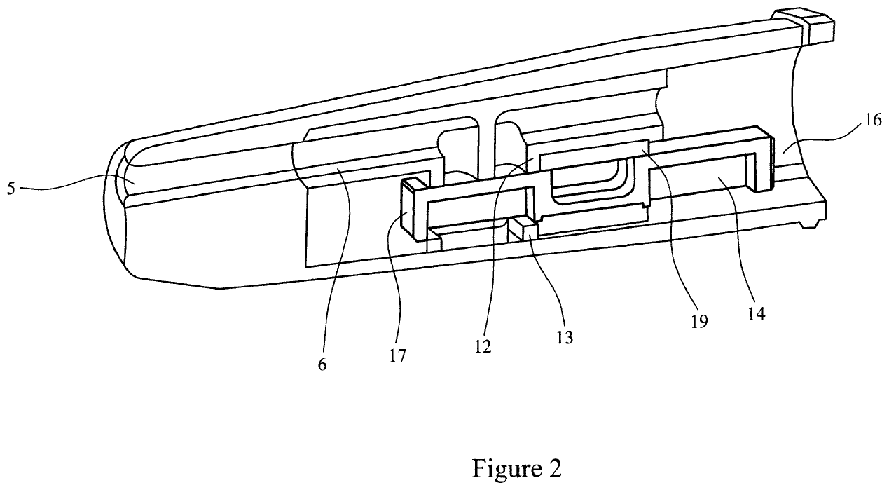

FIG. 2 illustrates the cartridge assembly 14 inserted into the body 4 such that the closure sleeve 19 abuts the cartridge facing lips 12 and 13.

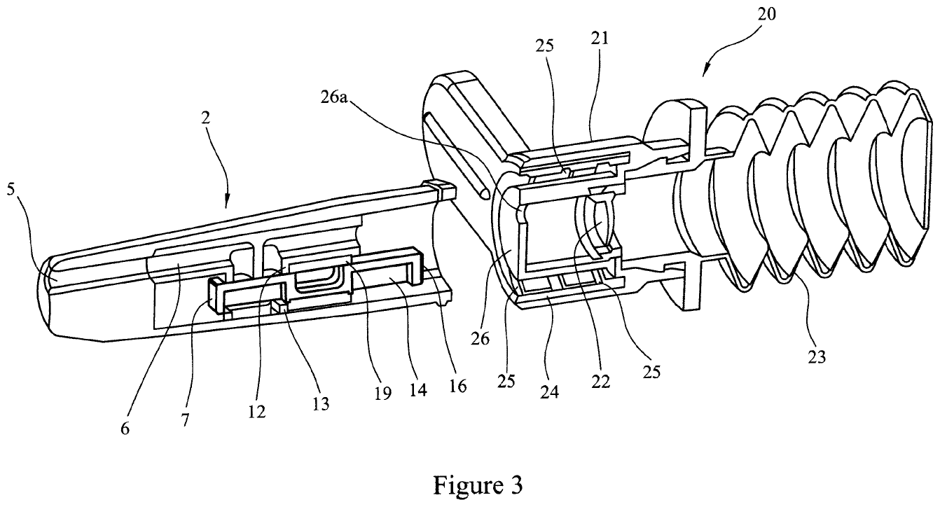

Referring to FIG. 3, the first element 2 is shown as described in FIGS. 1 and 2. Also shown is a second element 20 comprising an adaptor element 21, a burst valve 22 and bellows 23. The adaptor element 21 comprises a cylinder 24 with an internal screw thread 25 in the form of the helical grooves and a spigot 26 provided with an aperture 26a.

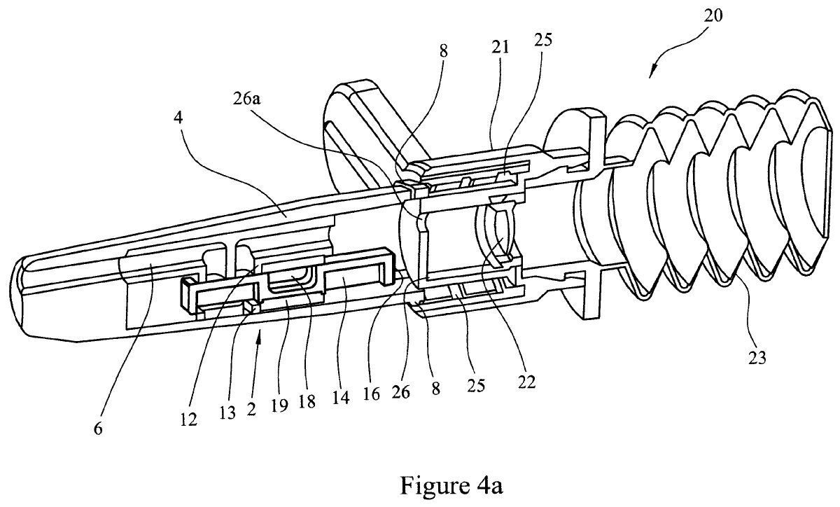

Referring to FIGS. 4a, 4b and 4c, in operation the second element 20 is attached to the first element 2, by locating the spigot 26 in the body 4 of the first element.

The circumferential rim 8 of the first element 2 is located into the screw thread 25 of the second element 20.

When a user is ready to administer medication, the device is “primed”. The body 4 of the first element 2 is held still and the second element 20 is rotated, screwing the adaptor element 21 onto the body 4 as the circumferential rim 8 is rotated in the helical grooves of the screw thread 25. As this is done, the spigot 26 engages with the first end wall 16 of the cartridge assembly 14 and pushes the cartridge assembly 14 within the body 4. As the adaptor element 21 is screwed further onto the body 4 the spigot 26 continues to engage with the first end wall 16 of the cartridge assembly 14. The closure sleeve 19 of the cartridge assembly engages with the cartridge facing lips 12 and 13 of the inner sleeve 6, causing the travel of closure sleeve 19 to stop whilst the (now open) cavity 18 continues to travel to the cartridge assembly seat 11, located under and aligned with the airway 9.

To administer the medicament (dry powder formulation) the bellows 23 is depressed to cause air pressure to build in bellows 23 to a point where the burst valve 22 opens releasing a burst of air into airway 9. The burst of air travels through the apertured spigot 26, down the airway 9 until it hits baffle 10 which deflects the air into the open cavity 18, thus entraining the dry powder formulation contained in the cavity 18 into the air stream and out via the airway 5 of the nozzle 3.

After administration, the first element 2 is unscrewed from the second element 20 and can be discarded. The second element 20, comprising an adaptor element 21 a burst valve 22 and bellows 23, can be retained for use in conjunction with a first element 2 and any subsequent elements.

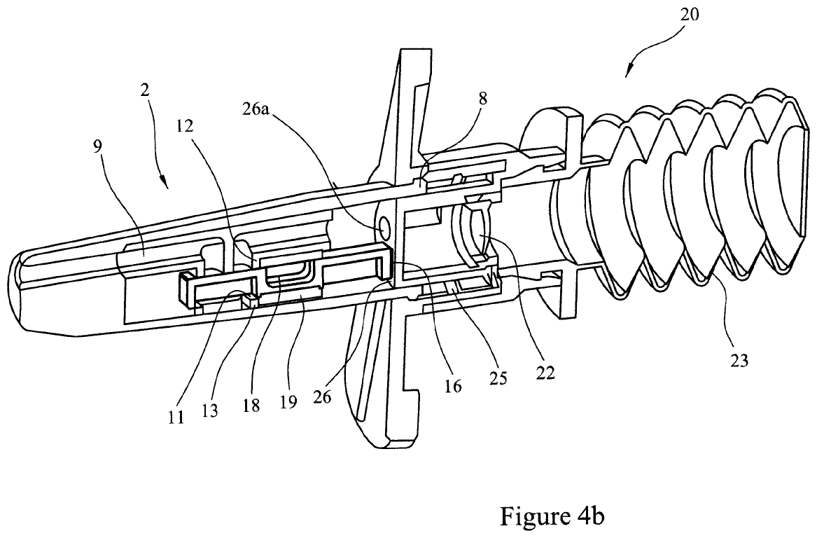

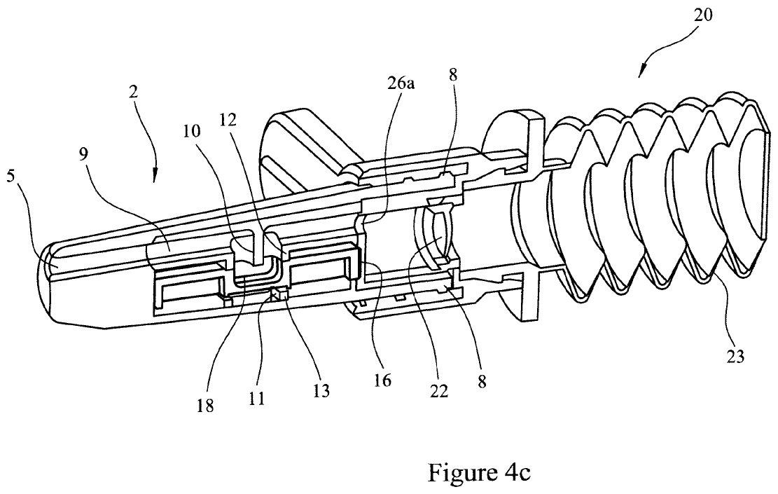

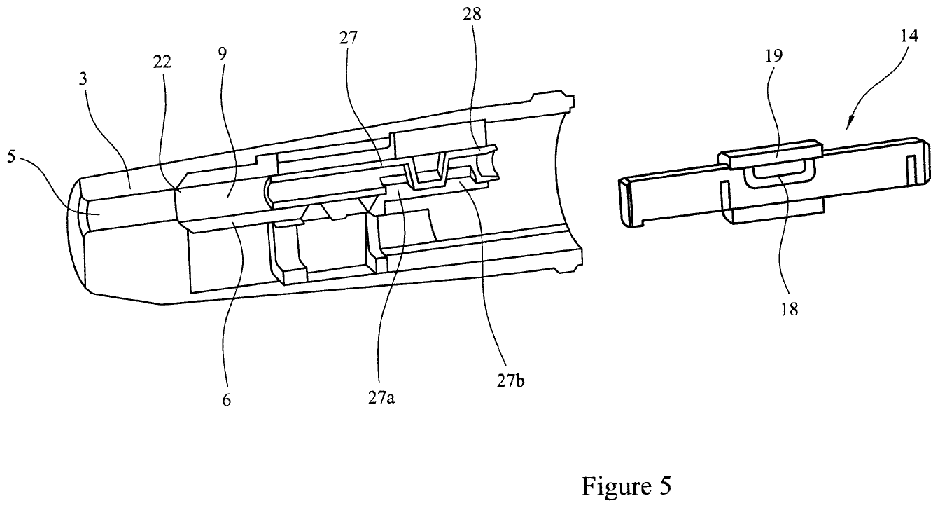

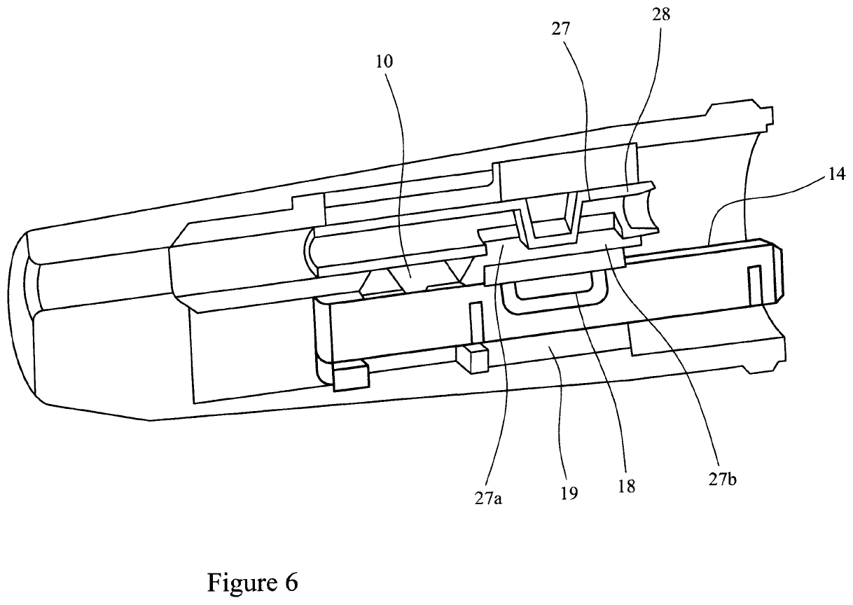

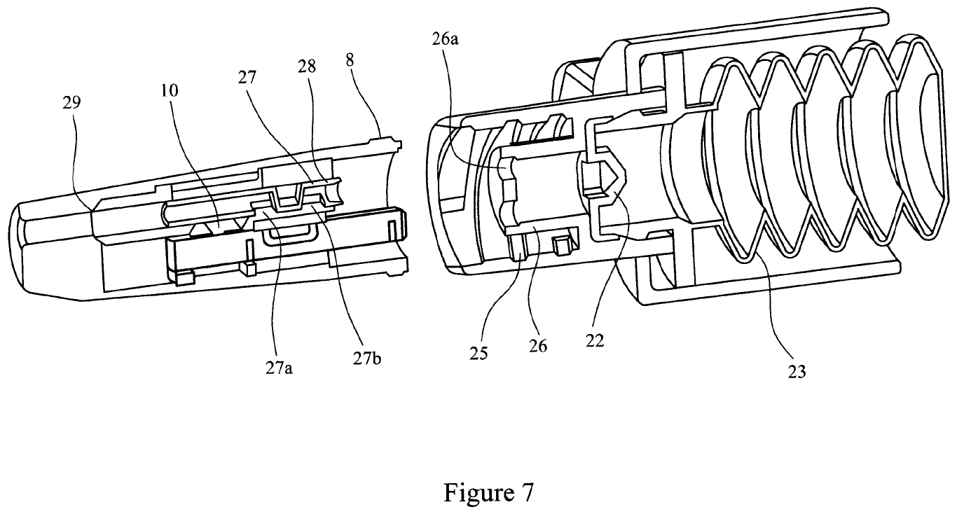

FIGS. 5, 6, 7, 8a, 8b, 8c and 9 illustrate a further embodiment of the multi-unit dose medicament delivery device. In this embodiment the airway 9 of the inner sleeve 6 is provided with a shuttle valve 27. The shuttle valve 27 is held in the closed position by a detent 28 in the inner sleeve 6. The shuttle valve 27 is dimensioned such that in the closed position it blocks both airway 9 and airway 5. In addition, the nozzle 3 is provided with a stop 29 capable of arresting movement of the shuttle valve 27. The shuttle valve 27 is provided with orifices 27a and 27b.

The use of a shuttle valve is advantageous in that it acts to prevent powder spillage, for example if the device is inverted and/or shaken after being “primed” but before the medicament is administered.

The operation of the multi-unit dose medicament delivery device of this embodiment is the same as herein described for FIGS. 1 to 4.

As the spigot 26 abuts the shuttle valve 27 it engages with it and pushes the shuttle valve 17 to release it from detent 28. At this point the airways 5 and 9 remain closed preventing any powdered medicament (not shown) from escaping from the device.

The procedure to administer the drug is as described above for the first embodiment. When air is released through the burst valve 22 the compressed air enters the shuttle valve 27 driving the shuttle valve 27 forwards to the stop 29. The orifices 27a and 27b of the shuttle valve 27 align with the airway 9, such that the air blast can then pass through the shuttle valve 27, airway 9, the medicament cavity 18, entraining the powdered medicament in the airflow, and through airway 5.

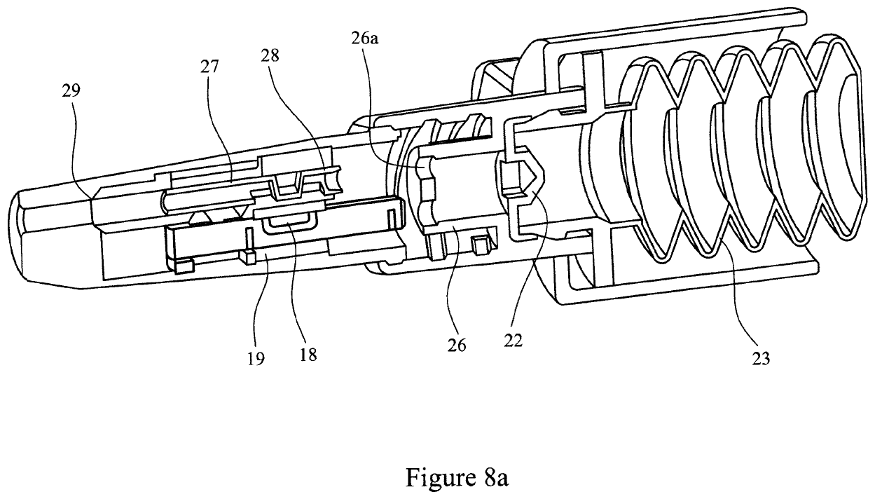

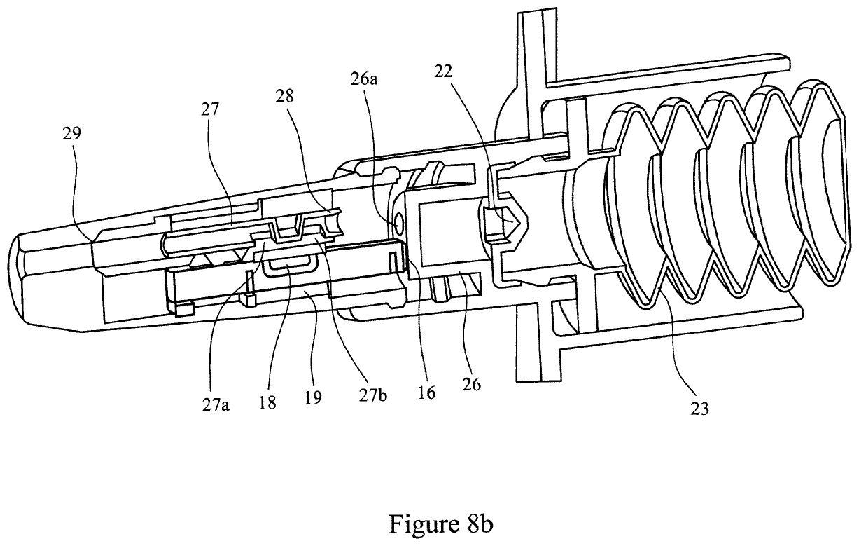

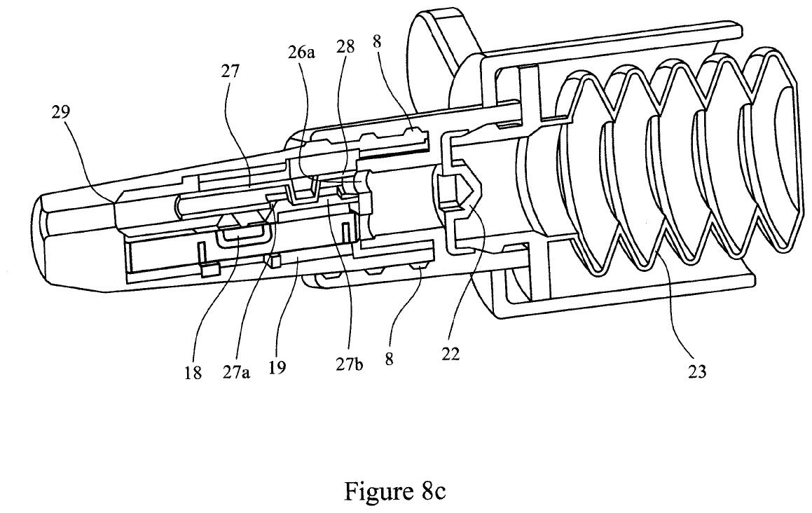

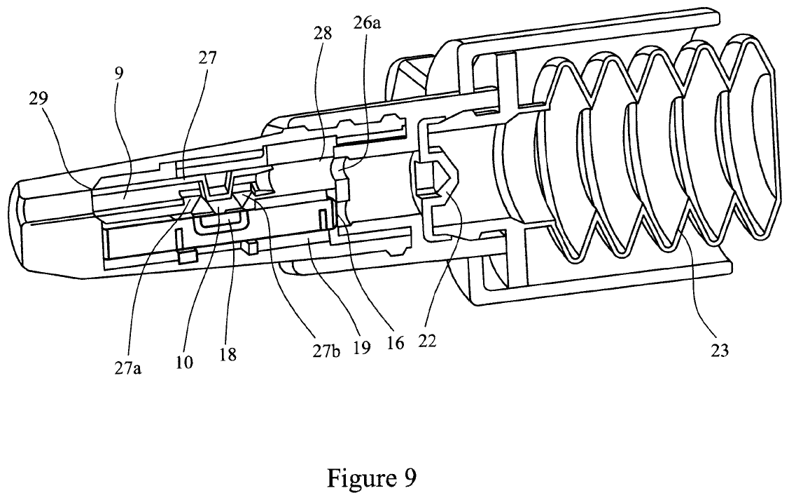

Referring to FIGS. 10 to 13 the burst valve 22 comprises a moulded flexible polymer disc 30 provided with a circumferential flange 31 to provide a sealing ring. Substantially in the centre of the disc there is provided a four sided pyramid structure 32 which forms a closure. The four sided pyramid structure 32 is provided with walls 33a, 33b, 33c and 33d. The pyramid structure 32 is provided with a slit 34 cut along a diagonal of the pyramid 32. In the valve's dormant state the slit 34 remains closed.

In the operation of the assembled device herein described (i.e. depressing the bellows 23), air pressure acts upon the outer surface of the pyramid 32 and walls (33a, 33b, 33c and 33d). The pyramid and wall structure is inherently a strong structure and the material (TPE) is compressed with the slit 34 being forced tightly dosed, however as pressure builds the pyramid is overcome and collapses and the slit 34 opens to create an orifice 35, allowing the compressed air to pass through the orifice 35. When the air pressure has diminished to ambient pressure, the pyramid 32 regains its moulded form. As the bellows 23 is released the outer surface of the pyramid 32 and walls (33a, 33b, 33c and 33d) are subjected to a negative pressure whilst the inner surfaces are subject to ambient pressure, the result is that the walls (33a, 33b, 33c and 33d) deflect outwardly relieving the forces on the slit 34, the effect being that air can ‘leak’ back into the bellows.

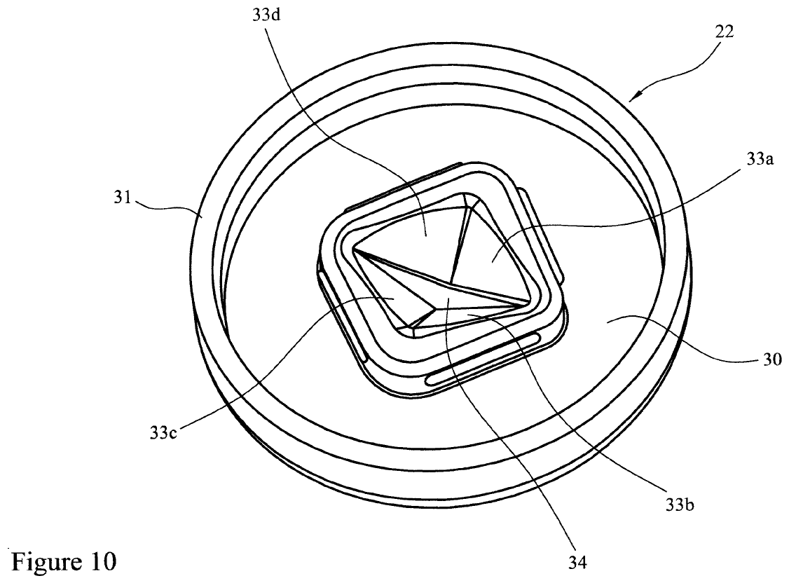

Referring to FIG. 14 is a multi-unit dose dry powder medicament delivery device 36 comprises a device body 37, an outer nozzle 38 at a first end 39, said outer nozzle 38 being provided with protective lid/dust cap 38a and a thumb catch 40; and a second end 41 being provided with plunger cap 42.

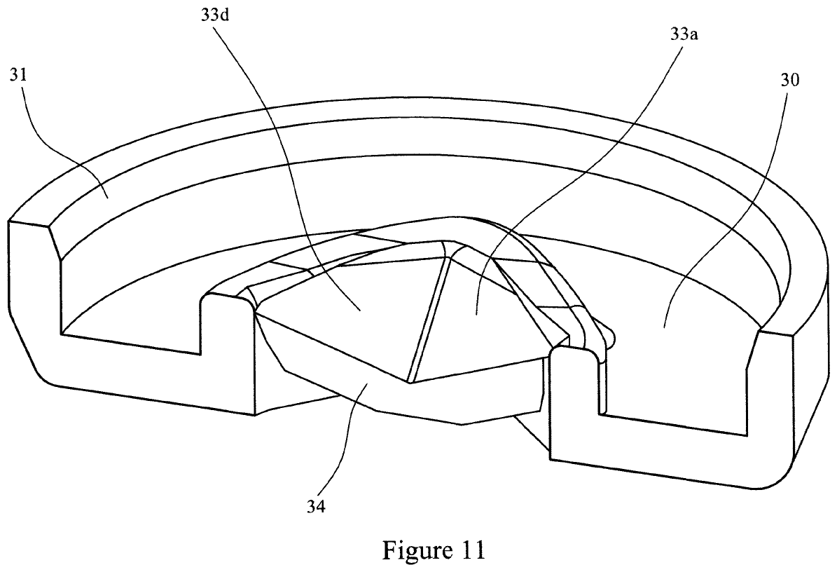

Referring to FIG. 15 is a multi-unit dose dry powder medicament delivery device 36 comprises a body 37, a plunger 43 and an outer nozzle 38. The plunger 43 is provided with a spigot 44 which is located within the body 37 of the device 36 and is positioned adjacent a delivery cartridge 45 located inside a sleeve 46 in the nozzle 38; said spigot 44 being provided with spring 47 which urges the spigot 44 away from the cartridge 45. The nozzle 38 is hinged to facilitate removal and replacement of the delivery cartridge 45.

The nozzle 38 is provided with a plunger retainer element 48 and a foil 49 is located between the spigot 44 and the delivery cartridge 45.

The plunger 43 is provided with a plunger cap 42 and a plunger latch 51a and 51b. A torsion spring 52 is located at one side 53 of the plunger cap 50. An O-ring seal 54 is located between the plunger 43 and the body 37. The nozzle end 55 of the delivery device 36 is provided with a hinge 56 on one side 57 allowing the nozzle 38 to be pivoted away from the body 37 of the delivery device 36.

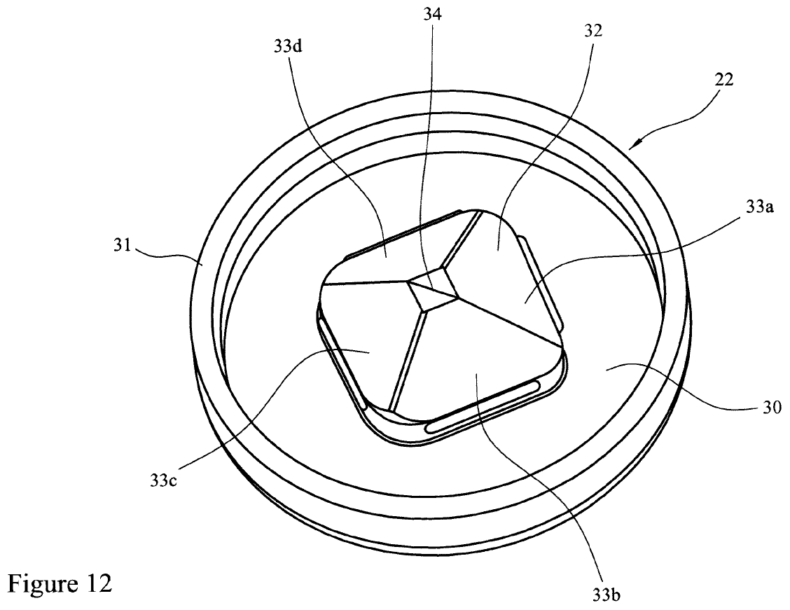

Referring to FIGS. 16 (a) and (b) a delivery device 36 is provided with replaceable delivery cartridge 45a and 45b shown prior to use in FIG. 16 (a) (foil 49a complete/not ruptured); and after use in FIG. 16 (b) (foil ruptured 49b). The nozzle 38 is pivotable about the hinge 56 to enable the delivery device 36 to be opened to facilitate removal of the spent cartridge 45b and insertion of a replacement (unspent) cartridge 45a.A cartridge seat 57 is provided in the nozzle 38.

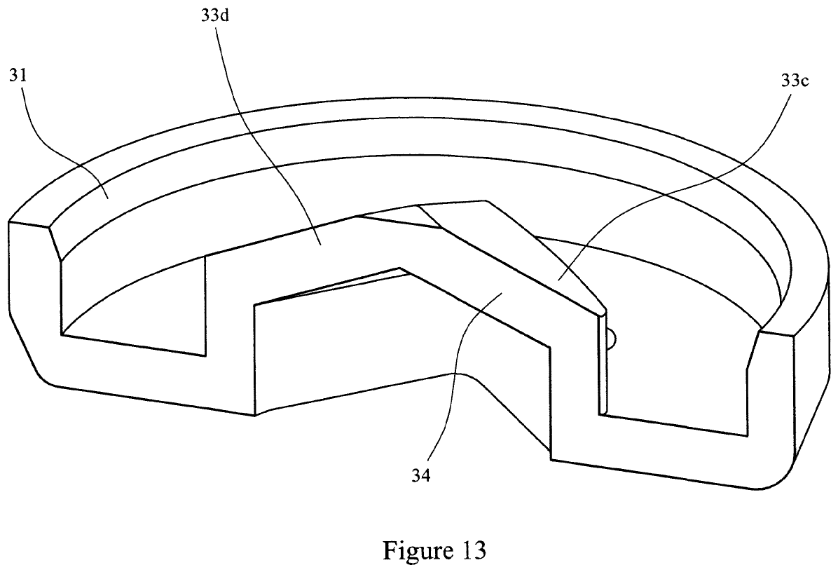

Referring to FIGS. 17 (a) and (b) a delivery device 36 is provided with a replaceable cartridge 45 (not shown in FIG. 17 (b)). In FIG. 17 (b) the nozzle 38 is snapped shut so that the delivery device 36 is ready for use.

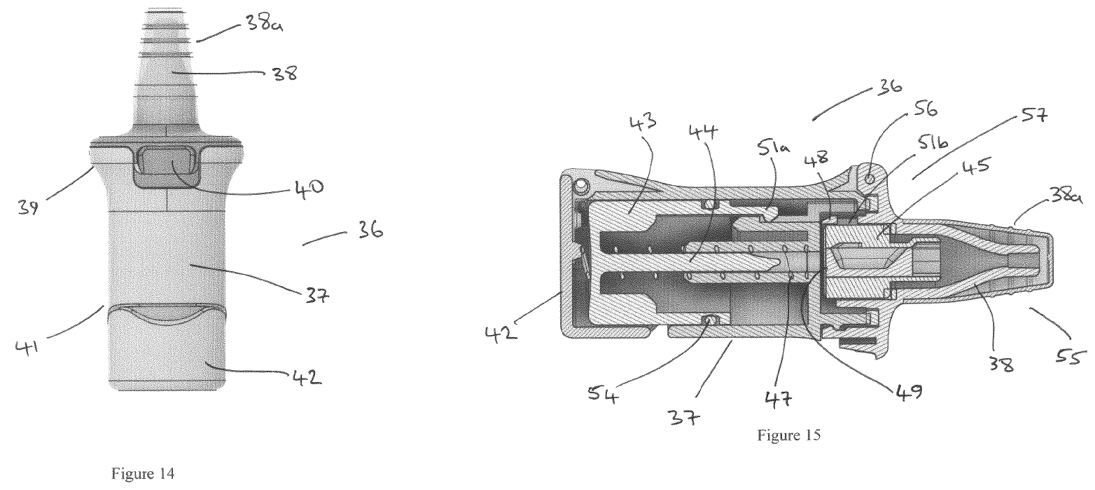

Referring to FIG. 18 is a replaceable cartridge 45 comprises three components, a carrier 58, a sleeve 59 and a foil 49. In use, the foil 49 is ruptured by the spigot (not shown) to indicate that the cartridge 45 has been used.

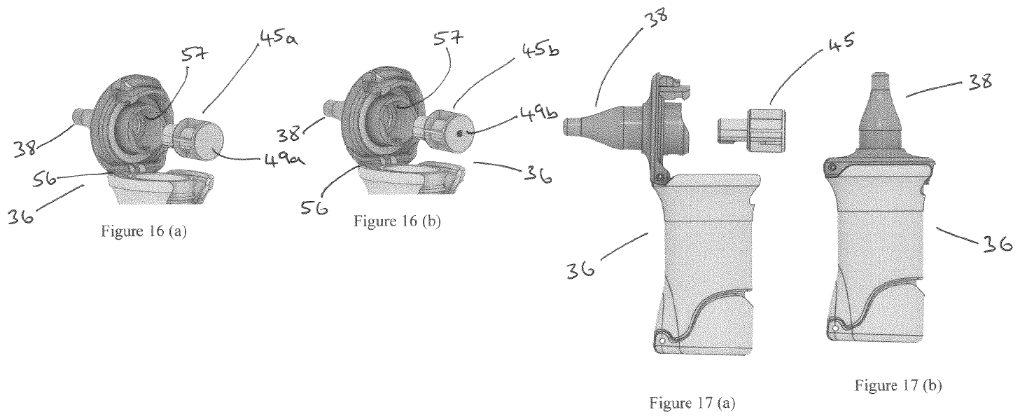

Referring to FIGS. 19 (a), (b) and (c); a delivery device 36 includes a plunger 43 wherein the end 60 of the plunger 43 adjacent to the nozzle 38 is provided with a latch mechanism 51a and 51b. The nozzle end 60 of the plunger 43 is provided with a resilient protrusion 62 which engages with a lip 63a or a groove 63b in the nozzle 38. The latch 51a and 51b retains the plunger 43 in place until the delivery device 36 is reset. This also provides the user with an indication that the device 36 has been activated correctly.

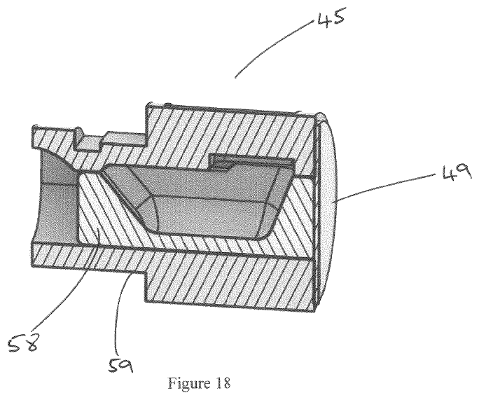

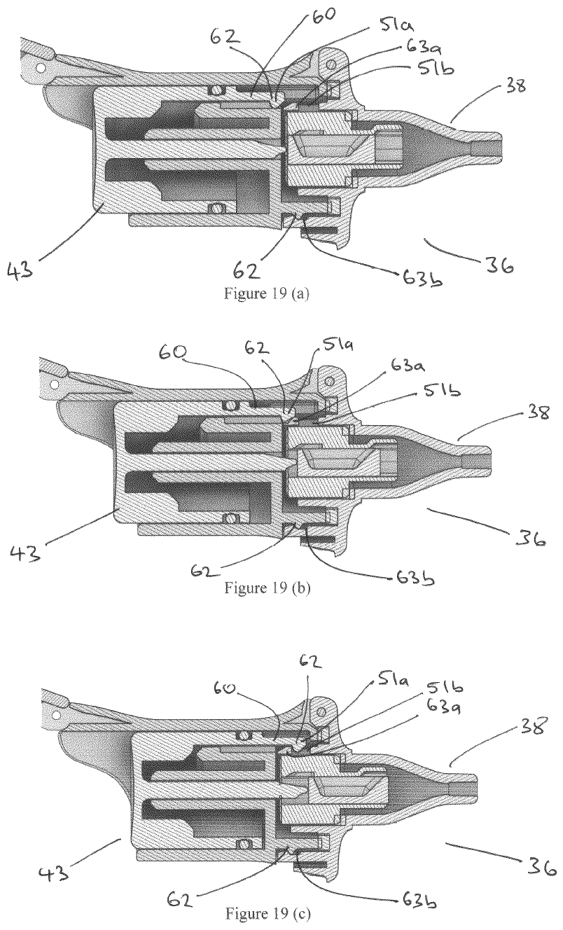

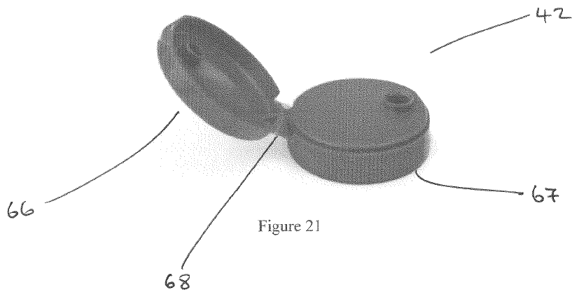

Referring to FIGS. 20 (a) to (d); in FIGS. 20 (a) and (b) a delivery device 36 is provided with a plunger cap 42, shown in the closed position in FIGS. 20 (a)-(c) and in the open position in FIG. 20(d). In FIG. 20 (b) a hinge 64 is shown, about which the plunger cap 42 can pivot. In FIG. 20 (c) the hinge 64 is shown with biasing means 65 in the form of a resilient torsion spring 65 mounted about the pivot. In FIG. 20 (d) the plunger cap 42 is showing in the open position, revealing the plunger 43.

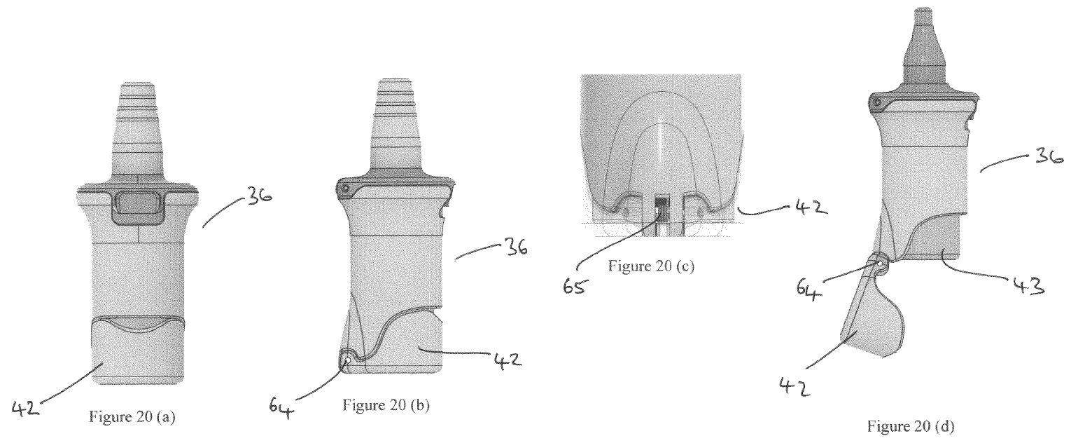

Referring to FIG. 21 a plunger cap 42 comprises a cover 66 and a base 67, such that the base 67 is adapted to fit over the end of the plunger (not shown). The cover 66 is attached to the base 67 by a resilient plastics hinge 68.

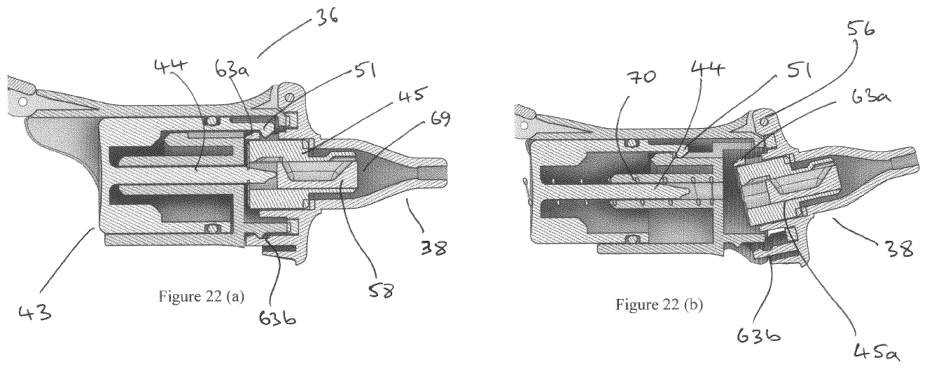

Referring to FIGS. 22 (a) and (b); FIG. 22 (a) illustrates the delivery device 36 in the actuated position, the plunger 43 is depressed such that the spigot 44 engages with the delivery cartridge 45 urging the medicament carrier 58 into the airway 69. The latch 51 of the plunger 43 is engaged with the lip 63a and/or groove 63b of the nozzle 38. Referring to FIG. 22 (b), after actuation, the spring 70 loaded spigot 44 is urged away from the nozzle 38 and the plunger latch 51 disengages from the lip 63a and/or groove 63b of the nozzle 38. The nozzle 38 is pivotable around the hinge 56 to allow access to the spent cartridge 45a which may be removed and the device recharged with a fresh (unused) cartridge (not shown).

1. A multi-unit dose dry powder medicament delivery device comprising:
a first element comprising a single use nozzle located in a body which is at least partially lined with an inner member, said inner sleeve comprising an airway and a cartridge seat; and
a second element adapted to be releasably attached to the first element, said second element comprising an actuator provided with an air source and a valve;
wherein the medicament cartridge comprises a replaceable cartridge.

2. A multi-unit dose dry powder medicament delivery device according to claim 1 wherein the replaceable medicament cartridge comprises three components; a medicament container (or carrier), a sleeve and a foil cover.

3. A multi-unit dose dry powder medicament delivery device according to claim 1 wherein the valve comprises the foil cover.

4. A multi-unit dose dry powder medicament delivery device according to claim 1 wherein the foil cover is positioned over one or both ends of the sleeve.

5. A multi-unit dose dry powder medicament delivery device according to claim 1 wherein the inner sleeve is provided with a shuttle valve.

6. A multi-unit dose dry powder medicament delivery device according to claim 5 wherein the shuttle valve is dimensioned such that in the closed position it blocks the airway only until the device is primed by a user.

7. A multi-unit dose dry powder medicament delivery device according to claim 6 wherein the shuttle valve is provided with a pair of orifices which, when a device is primed, align with the airway of the device.

8. A multi-unit dose dry powder medicament delivery device according to claim 6 wherein the pair of orifices comprise an entrance orifice, enabling air to be drawn into the device; and an exit orifice, allowing air and the entrained medicament, to be drawn into the medicament carrier.

9. A multi-unit dose dry powder medicament delivery device according to claim 1 in conjunction with a cartridge comprising a dry powder medicament.

10. A multi-unit dose dry powder medicament delivery device according to claim 9 wherein the medicament cartridge comprises a unit dose of drug or medicament.

11. A multi-unit dose dry powder medicament delivery device according to claim 1 wherein the actuator in the second element comprises a spigot element which, when inserted into the first element, pushes the medicament container into position for delivery of the medicament.

12. A multi-unit dose dry powder medicament delivery device according to claim 11 wherein the spigot element is provided with at least one aperture to enable air to pass from the air source and valve through the spigot.

13. A multi-unit dose dry powder medicament delivery device according to claim 11 wherein the actuator comprises a plunger, such that, in use, a user presses or depresses the plunger in order to actuate the device and urge the spigot toward the cartridge assembly.

14. A multi-unit dose dry powder medicament delivery device according to claim 13 wherein the delivery device is provided with a removable protective cap or cover adapted to be positioned over the plunger.

15. A multi-unit dose dry powder medicament delivery device according to claim 14 wherein the protective cap is held in the closed (protective) position by the use of biasing means.

16. A multi-unit dose dry powder medicament delivery device according to claim 15 wherein the biasing means comprises a torsion spring.

17. A multi-unit dose dry powder medicament delivery device according to claim 16 wherein the biasing means comprises an integral moulded hinge which acts to urge the protective cap to the closed (protective) position.

18. A multi-unit dose dry powder medicament delivery device according to claim 13 wherein the actuator or plunger is provided with a latch mechanism, such that the latch retains the plunger in place after the device has been actuated until the device is reset by.

19. A multi-unit dose dry powder medicament delivery device according to claim 18 wherein the latch mechanism comprises a resilient protrusion adapted to releasably engage with a portion of the first element of the delivery device.

20. A multi-unit dose dry powder medicament delivery device according to claim 18 wherein the first element of the delivery device may be provided with one or more grooves adapted to releasably engage with the plunger latch.

21. A multi-unit dose dry powder medicament delivery device according to claim 11 wherein the device comprises a dynamic relationship between the spigot, the entrance orifice and the medicament carrier.

22. A multi-unit dose dry powder medicament delivery device according to claim 21 wherein, in use, as the spigot progressively moves the carrier forward within the sleeve, the entrance orifice is progressively revealed.

23. A multi-unit dose dry powder medicament delivery device according to claim 1 wherein the device is a nasal inhaler.

24. A multi-unit dose dry powder medicament delivery device according to claim 1 wherein the delivery device is suitable for the delivery of an emergency therapy.

25. A multi-unit dose dry powder medicament delivery device according to claim 24 wherein the delivery device is for the nasal delivery of an emergency therapy.

26. A multi-unit dose dry powder medicament delivery device according to 24 wherein the dry powder medicament delivery device is for the delivery of a therapeutically effective amount of glucagon to a diabetic experiencing a hypoglycaemic reaction.

27. A multi-unit dose dry powder medicament delivery device kit comprising:
a first element comprising a single use nozzle, said nozzle being located on a body which is at least partially lined with an inner sleeve, said inner sleeve comprising an airway and a cartridge seat;
a second element adapted to be releasably attached to the first element, said second element comprising an actuator provided with an air source and a valve;
wherein the medicament cartridge comprises a replaceable cartridge and
at least one cartridge comprising a dry powder medicament.

28. The kit according to claim 27 wherein the inner sleeve is provided with a shuttle valve and said shuttle valve is dimensioned such that in the closed position it blocks the airway only until the device is primed by a user.

29. The kit according to claim 27 which includes a plurality of medicament cartridges.

30. A method of delivering a medicament to a patient which comprises the use of a dry powder medicament delivery device according to claim 1.

31. A method of treatment of a patient with a disorder or prevention of a disorder which comprises the administration to the patient a suitable medicament using a medicament delivery device according to claim 1.
